(19) United States
(12) Patent Application Publication
(10) Pub. No.: US 2012/0178947 A1
(43) Pub. Date: Jul. 12, 2012

(54) PROCESS FOR HYDROGENATING 1,4-BUTYNEDIOL TO GIVE MIXTURES COMPRISING TETRAHYDROFURAN, 1,4-BUTANEDIOL AND GAMMA-BUTYROLACTONE IN THE GAS PHASE

(75) Inventors: Rolf PINKOS, Bad Duerkheim (DE); Olga Osetska, Mannheim (DE); Lucia Königsmann, Stuttgart (DE)

(73) Assignee: BASF SE, Ludwigshafen (DE)

(21) Appl. No.: 13/346,243

(22) Filed: Jan. 9, 2012

Related U.S. Application Data
(60) Provisional application No. 61/431,875, filed on Jan. 12, 2011.

(51) Int. Cl. C07D 307/20; C07C 29/17; C07D 307/08

(52) U.S. Cl. 549/475; 549/509; 568/861

(57) ABSTRACT

Process for preparing tetrahydrofuran, 1,4-butanediol and/or gamma-butyrolactone by hydrogenation of 1,4-butynediol, wherein 1,4-butynediol is vaporized in a hydrogen-comprising gas stream and hydrogenated in gaseous form over at least one catalyst which comprises at least one of the elements of groups 7 to 11 of the Periodic Table of the Elements.

The present application incorporates the prior U.S. application Ser. No. 61/431,875 submitted on Jan. 12, 2011, by reference.

The process of the invention relates to the catalytic hydrogenation of 1,4-butynediol to give mixtures comprising tetrahydrofuran (THF), 1,4-butanediol (BDO) and/or gamma-butyrolactone (GBL) in the gas phase over heterogeneous catalysts in the presence of a hydrogen-comprising gas.

THF is widely used as solvent and serves as starting material for polytetrahydrofuran. It is produced in several hundred thousand metric tons per year worldwide.

BDO is a sought-after diol, for example for preparing polyesters or polyurethanes, and GBL serves industrially as solvent or is used as intermediate for preparing pyrrolidones. The worldwide most widespread technology employed industrially for preparing 1,4-butanediol is the hydrogenation of 1,4-butynediol under a substantial pressure in the range from 20 to 300 bar, known as the Reppe technology which is described, for example, in DE-A 19641707. Here, it has to be noted in the reaction of 1,4-butynediol that the latter can decompose under thermal stress, especially in the presence of impurities, in particular metals and salts thereof. In the case of the pure material, this generally occurs at and above 160° C., in particular above 200° C. Impurities, in particular heavy metals, can further reduce this decomposition temperature.

THF can be obtained from 1,4-butanediol by the acid-catalyzed cyclization of 1,4-butanediol, as described, for example, in WO-A 2005/87757. GBL can likewise be prepared from 1,4-butane-diol, for example by catalytic dehydrogenation, as described by K. Weissermel, H.-J. Arpe, Industrielle Organische Chemie, 5th edition 1998, pages 112 to 114. A disadvantage is that for these reactions to form THF and GBL, 1,4-butanediol firstly has to be isolated and in general has to be purified.

The preparation of THF and GBL from 1,4-butanediol which has been obtained by hydrogenation of 1,4-butynediol is therefore an industrially established route, but it would be desirable, for example because of lower capital costs, for the hydrogenation of 1,4-butynediol to give THF, BDO and GBL directly without 1,4-butanediol having to be isolated as intermediate.

Processes described for the direct preparation of THF from butynediol are all carried out in the liquid phase. These are at present not carried out industrially. DE-A 2029557 describes a process for the direct preparation of THF from 1,4-butynediol in the liquid phase. According to the disclosure of DE-A 2029557, butynediol is reacted in the liquid phase in the presence of a solvent over a catalyst having a hydrogenation function and acidic function to form THF and water. Reworking the examples shows that the process cannot be carried out. Mainly butanediol was found as product, but only negligible amounts of THF.

It is therefore an object of the present invention to provide a process for the catalytic hydrogenation of 1,4-butynediol to form BDO, THF and GBL, which process displays good selectivities and long catalyst operating lives and thus makes possible a direct route to THF, BDO and GBL from 1,4-butynediol which can be carried out industrially because of its good economics.

It has now surprisingly been found that BDO, THF and GBL can be obtained in high yield by catalytic hydrogenation of 1,4-butynediol at at least the decomposition temperature of 1,4-butynediol by vaporizing 1,4-butynediol in a hydrogen-comprising gas stream and hydrogenating it in gaseous form over at least one catalyst which comprises at least one of the elements of groups 7 to 11 of the Periodic Table.

The temperature for vaporization and the hydrogenation temperature are in the range 160-330° C., preferably 160-300° C., particularly preferably 170-280° C. The temperature at which the 1,4-butynediol is vaporized can, in the abovementioned temperature range, be lower than the hydrogenation temperature. The pressure during vaporization and hydrogenation is from 0.05 MPa (megapascal) to 10 MPa absolute, preferably from 0.1 to 6 MPa absolute, particularly preferably from 0.15 to 2 MPa absolute. The pressure during vaporization corresponds to at least the pressure in the hydrogenation.

1,4-Butynediol can be used as pure material, but preference is given to using 1,4-butynediol as it is obtained from the synthesis stage for 1,4-butynediol. This technical-grade butynediol can comprise, for example, water, propynol, formaldehyde in free form or bound as hemiacetals or acetals, methanol and also small amounts, in general less than 1%, of acetylene, dissolved or solid materials such as catalyst constituents from the 1,4-butynediol synthesis catalyst (e.g. copper salts such as copper acetylides which increase the decomposition susceptibility of butynediol) or salts which originate from a pH regulation, e.g. sodium formate, oligomeric or polymeric secondary components (known as cuprenes), C₅ and C₆ components which can be attributed to impurities in the acetylene used or to secondary reactions in the butynediol synthesis. When no pure butynediol which can be obtained from technical-grade 1,4-butynediol, for example by distillation, is used, the water content in the starting material is from 5 to 89% by weight, particularly preferably from 30 to 70% by weight. The 1,4-butynediol content is generally from 10 to 90% by weight, preferably from 20 to 80% by weight, particularly preferably from 30 to 70% by weight. The propynol content is generally below 10% by weight, preferably below 5% by weight, particularly preferably below 3% by weight. Formaldehyde calculated as the sum of formaldehyde itself, hydrate, acetal or hemiacetal is present in an amount of less than 5% by weight, preferably less than 2% by weight, particularly preferably less than 1% by weight. The methanol content is below 50% by weight, preferably below 5% by weight, particularly preferably below 1% by weight. The content of nonvolatile constituents is generally below 2% by weight, preferably below 1% by weight, particularly preferably below 0.1% by weight.

The vaporization of the 1,4-butynediol-comprising stream is generally carried out at pressures which correspond to at least the later hydrogenation pressure. However, it is also possible to choose a higher pressure, for example a pressure which is up to 0.5 MPa higher, in the vaporization of the 1,4-butynediol. Such an increased pressure can compensate for pressure drops which occur, for example as a result of pipes, valves, catalysts, heat exchangers.

The vaporization of the 1,4-butynediol is carried out in the presence of a hydrogen-comprising gas in apparatuses which are known per se for vaporization, for example in one or more falling film evaporators, thin film evaporators, helical tube evaporators, single-fluid or multifluid nozzles, pipes filled with inert material in cocurrent or countercurrent with hydrogen, natural convection vaporizers, forced circulation vaporizers, kettle-type vaporizers or steam boilers. The 1,4-butynediol-comprising gas stream can be heated further in order to attain the desired reactor inlet temperature of the hydrogenation reactor.

As medium for introducing heat into the vaporization, it is possible to use an appropriately preheated hydrogen-comprising gas stream which can comprise hydrogen together with helium, nitrogen and carbon dioxide and, if recycle gas from the hydrogenation is used, methane, ethane, propane, butane, methanol, dimethyl ether, ethanol, propanol, butanol, carbon monoxide, THF and water. These components are present in the gas stream in a proportion by mass which is generally below 50%, preferably below 40%, preferably below 30%. However, it is also possible to produce indirect heat exchange, for example by means of electric heating or heat transfer media such as steam or oil.

Components having a boiling point higher than butynediol can be added to the 1,4-butynediol-comprising feed stream to the vaporization, for example when high boilers such as cuprenes and salts are present in the butynediol-comprising stream. These high-boiling components prevent solidification of, for example, the salts in the vaporizer. These high boilers can be, for example, alcohol-, ester-, ether-, urea-, urethane-, amide-comprising substances. Mention may be made by way of example of glycerol and oligomers thereof and Sokalan grades. The high boilers, which based on butynediol can be added in amounts of from 0.001 to 5% by weight, are then discharged together with the salts and cuprenes from the 1,4-butynediol and preferably burnt to generate energy.

The catalyst used in the process of the invention has at least one element of groups 7 to 11 of the Periodic Table of the Elements as active components for the hydrogenation. These elements can be present in the form of one or more metals or in the form of low-valence compounds of these metals, for example as oxides which are likewise hydrogenation-active. Of the hydrogenation-active elements of groups 7 to 11 of the Periodic Table of the Elements, the catalyst preferably comprises Mn, Re, Fe, Ru, Co, Rh, Ir, Ni, Pd, Pt, Cu and/or Au, particularly preferably Ru, Rh, Ir, Ni, Pd and/or Pt, in particular Pd, Ni and/or Cu, as active component(s) from groups 7 to 11 of the Periodic Table of the Elements. M. M. Telkmar et al., Journal of Molecular Catalysis A: Chemical (2002), 187(1), 81-93 and R. Chaudhari et al. Applied Catalysis (1987), 29(1), 141-59 disclose that catalysts based on palladium are preferably used in the liquid-phase hydrogenation when incomplete-conversion of the 1,4-butynediol in the hydrogenation is desired and 1,4-butenediol is the desired product. It is therefore surprising that palladium catalysts according to the process of the invention lead directly to THF, BDO and GBL in the gas phase.

The metal content (active component) of the catalysts which can be used in the process of the invention is generally in the range from 0.001 to 100% by weight. In the case of catalysts which are generally produced by metal salt precipitation, the metal content is from 1 to 100% by weight, preferably from 5 to 90% by weight, particularly preferably from 10 to 80% by weight. In the case of catalysts which are produced by impregnation, the metal content is from 0.001 to 50% by weight, particularly preferably from 0.01 to 20% by weight, particularly preferably from 0.1 to 10% by weight.

The catalysts which are used in the process of the invention can additionally comprise at least one element or element compound selected from among the elements of groups 1 to 16 of the Periodic Table of the Elements and the lanthanides. These elements or element compounds can be comprised as a result of the method of manufacture or can also be added deliberately, for example as promoter for the reaction and/or as support for the active component. The catalyst used in the process of the invention is preferably a supported catalyst.

The promoter content of the catalyst can be up to 25% by weight, preferably from 0.001 to 15% by weight, particularly preferably from 0.01 to 13% by weight. Mention may be made by way of example of alkali metal or alkaline earth metal components such as hydroxides, oxides, carbonates or salts of organic or inorganic acids, e.g. in order to vary the basic properties of the catalysts. Furthermore, sulfur, phosphorus, silicon and aluminum components can serve to modify the acidic properties of the catalysts. For example, sulfuric acid or phosphoric acid can be anchored on the catalyst. Particularly in the case of catalysts based on carbon, e.g. activated carbons, the acidic property of the support can also be adjusted by treating the carbon with, for example, hydrochloric, sulfuric, phosphoric or nitric acid. This can occur before or after impregnation with active component, preferably before.

As catalysts, it is possible to use precipitated, supported or Raney-type catalysts, the production of which is described, for example, in Ullmanns, Encyclopädie der technischen Chemie, 4th edition, 1977, volume 13, pages 558-665.

As support materials of the supported catalysts which are preferably used in the process of the invention, it is possible to use, for example, aluminum oxides, titanium oxides, zirconium dioxide, silicon dioxides, silicon carbide, sheet silicates, clay minerals, e.g. montmorillonites, silicates such as magnesium or aluminum silicates, zeolites and also activated carbons. Preferred support materials are aluminum oxides, titanium dioxides, silicon dioxide, zirconium dioxide and activated carbons. Of course, mixtures of various support materials can also serve as support for catalysts which can be employed in the process of the invention.

These supports can also be prefabricated monoliths, e.g. monoliths made of ceramic, SiO₂, Al₂O₃, etc., or, for example, corrugated sheets which are later rolled up so as to give a cylindrical shape through which fluid can flow or, for example, wire knitteds which can likewise be shaped.

Suitable catalysts for the hydrogenation according to the invention of 1,4-butynediol to THF are heterogeneous catalysts which are preferably used as shaped bodies. For the purposes of the present patent application, shaped bodies are, for example, crushed material, extrudates and pellets. These shaped bodies can also be hollow bodies, for example hollow cylinders, stars and trilobes in order to increase the surface area. Preference is given to using catalysts in the form of crushed material, pellets and extrudates in the process of the invention. These shaped bodies have diameters of from 0.1 to 20 mm. Preference is given to from 1 to 10 mm, particularly preferably from 1.5 to 7 mm. The length of the catalyst bodies is not critical, but should generally be no less than the diameter. Preferred lengths of the shaped catalyst bodies are from 1 to 50 mm.

The supported catalysts used according to the invention are produced by applying the active component or the combinations of active components which can be applied together or in succession. Application can be effected by methods known per se, for example by impregnation, precipitation, sputtering. The active components are generally present as a thin layer on the support. In the case of application by sputtering, the content of active material based on the support can also be below 0.001% by weight. In this case, a content of active component of from 0.00001 to 0.5% by weight is preferred.

Further suitable catalysts for the process of the invention are Raney-type catalysts, for example Raney nickel, Raney copper, Raney cobalt, Raney nickel/molybdenum, Raney nickel/copper, Raney nickel/chromium, Raney nickel/chromium/iron, Raney nickel/palladium or rhenium sponge. Raney nickel/molybdenum catalysts can, for example, be produced by the process described in U.S. Pat. No. 4,153,578. However, these catalysts are also marketed by, for example, Degussa, 63403 Hanau, Germany. A Raney nickel-chromium-iron catalyst is, for example, marketed under the trade name Katalysator type 11 112 W® by Degussa.

The Raney catalysts are likewise preferably used as shaped bodies, e.g. pelletized or extruded, but it is likewise possible to treat granulated alloy with, for example, sodium hydroxide solution so that only an outer layer of the particle is leached out and exposes the active Raney layer. Such particles then have, for example, diameters in the range from 1 to 10 mm.

When precipitated or supported catalysts are used, these are preferably reduced in a hydrogen or hydrogen/inert gas stream at from 20 to 500° C. before commencement of the reaction; this reduction can, for example, be carried out in the presence of hydrogen during heating-up of the reactor to the start temperature. The reduction temperature depends on the desired degree of reduction and the temperature necessary for the active component. Thus, Pd present, for example, as PdO on a support requires temperatures of from 20 to 100° C., while Co oxides require from 200 to 300° C. for activation by means of hydrogen. This reduction can be carried out directly in the hydrogenation reactor. If the reduction is carried out in a separate reactor, the catalysts can be passivated on the surface by means of oxygen-comprising gas mixtures at, for example, 30° C. before removal from the reactor. The passivated catalysts can in this case be activated in a stream of nitrogen/hydrogen at, for example, 180° C. in the hydrogenation reactor before use or be used without activation.

It is possible to use one type of catalyst for the process of the invention. However, it is also possible to use mixtures of a plurality of catalysts. These mixtures can be present as pseudohomogeneous mixture or as a structured bed in which individual reaction zones each composed of a pseudohomogenous catalyst bed are present. It is also possible to combine the methods, i.e., for example, to use one catalyst type at the beginning of the reaction and to use a mixture further downstream.

In a preferred embodiment of the process of the invention, when THF is to be the preferred product, an acidic catalyst which has no hydrogenation properties but is able to convert 1,4-butanediol into THF and water is used in addition to at least one of the above-described catalysts which has at least one of the elements of groups 7 to 11 of the Periodic Table of the Elements as active component for the hydrogenation. In this particular embodiment of the process of the invention, aluminum oxides, titanium oxides, zirconium dioxides, silicon dioxides, sheet silicates, clay minerals, e.g. montmorillonites, silicates such as magnesium or aluminum silicates, zeolites and also acidified activated carbons can be used as acidic catalyst. To increase the acidity, it is possible to employ the usual methods, e.g. application of sulfuric or phosphoric acid. It is important that no basic components such as amines or possibly volatile or entrained salts which can neutralize the acidic sites get onto the acidic catalyst.

There are a number of possibilities for industrial implementation of the hydrogenation. After vaporization of the 1,4-butynediol or the 1,4-butynediol-comprising stream, preferably by means of a stream of hydrogen and heating, the butynediol-comprising gas stream goes into the hydrogenation reactor. After the hydrogenation, the gas stream is cooled, the product is largely separated from hydrogen and the product stream is worked up further. The remaining hydrogen is partly discharged and part is recirculated, preferably as recycle gas.

The hydrogenation is carried out using a hydrogen-comprising gas stream which can comprise hydrogen together with helium, nitrogen and carbon dioxide in a proportion by mass of the gas stream which is generally below 50%, preferably below 40%, preferably below 30%.

The space velocity over the catalyst in the hydrogenation according to the invention is generally from 0.01 to 3 kg of 1,4-butynediol/(l of catalyst•h). Preference is given to space velocities over the catalyst of from 0.05 to 2 kg of 1,4-butynediol/l of catalyst•h, particularly preferably from 0.1 to 1 kg of 1,4-butynediol/l of catalyst•h.

The molar ratio of hydrogen to be consumed chemically by hydrogenation to 1,4-butynediol used is at least 1.5:1, preferably 2-4:1 for advantageous reaction. After the reaction, excess hydrogen can be discharged. It is preferable to have a high molar ratio of hydrogen to butynediol or reaction products thereof, for example 4-400:1, preferably 20-300:1, particularly preferably 40-200:1, during vaporization or during the reaction.

This is achieved by the preferred gas recycle mode of operation in which at least part of the hydrogen or the hydrogen-comprising gas stream is circulated. The amount of hydrogen consumed chemically by the hydrogenation and by discharge is replaced. In a preferred embodiment, part of the recycle gas is discharged in order to remove inert compounds. The circulated hydrogen-comprising gas can also be utilized for vaporizing the 1,4-butynediol stream in the process of the invention.

The proportion of hydrogen discharged, calculated as proportion of hydrogen consumed chemically by hydrogenation, is from 100 to 0.1%, with preference being given to from 50 to 0.2%, particularly preferably from 20 to 0.3%. The lower this proportion, the less hydrogen is discharged and the more economical is the process. The discharge can be effected firstly by means of offgas, secondly via the proportion of hydrogen dissolved in the cooled, liquid product stream. The discharge is carried out in order to discharge inerts or secondary components in a targeted manner. Inerts can, for example, be introduced by the hydrogen, e.g. He, N₂, CO₂. Secondary components can be, for example, methane, ethane, propane, butane, methanol, dimethyl ether, ethanol, propanol, butanol and carbon monoxide which are formed in the reaction. Further components in the recycle gas stream can be desired reaction products such as BDO, GBL and THF and also water. It is advantageous for the inerts, products, water and secondary components not to be present in too high a concentration in the recycle gas since they reduce the partial pressure of hydrogen. They should have a proportion of less than 50% in the recycle gas. A special case is carbon monoxide, possibly also carbon dioxide, since these can reduce the activity of the active components. The proportion of carbon monoxide and/or carbon dioxide in the recycle gas should therefore ideally be below 10%, preferably below 5%, particularly preferably below 1%.

The temperatures in the hydrogenation reactor are preferably maintained so that the temperature increases along at least ¼ of the length of the catalyst bed, independently of the reactor type used. If cooling were to be brought about along the catalyst bed, especially in the first zone of the catalyst, there would be a risk of condensation which would then lead to deactivation of the catalyst by formation of carbon deposits. The temperature increase along at least the first quarter of the catalyst bed is 1-100° C., preferably from 2 to 80° C., particularly preferably from 5 to 60° C. After the maximum temperature has been reached, the reaction temperature can, depending on the reactor type (e.g. shell-and-tube reactor), drop again. Here, the inlet temperature is the temperature of the gas stream at which this stream impinges on the catalyst.

After leaving the catalyst bed or the hydrogenation reactor, the gas stream (hydrogenation output) is cooled in one or more stages to such an extent that a liquid phase, viz. the product mixture, is formed. This preferably occurs at the same pressure level as the reaction itself. Cooling can be effected by means of air or water coolers, refrigeration plants or other industrial auxiliaries. The condensation temperature is generally in the range from −78° C. to 50° C., preferably from −15° C. to 40° C., particularly preferably from −10° C. to 30° C.

A considerable advantage of the process of the invention is that THF comprised in the recycle gas does not decompose or decomposes only very insignificantly. It has surprisingly been found that THF remains unchanged to an extent of at least 99% even during a second pass through the catalyst. Although complete condensation of the product is desirable, the minimum condensation temperature required for condensation of the product stream is not critical because of the above-described stability of the THF, as a result of which energy can be saved.

As reactor or types of reactor for the hydrogenation according to the invention in the gas phase, it is possible to use, for example, shell-and tube reactors, shaft reactors or fluidized-bed reactors. A particular type would be the microreactor which is particularly advantageous when the heat of the reaction is to be removed very efficiently in order to keep the temperature of the reaction as constant as possible. The individual reactors or types of reactor can also be used as a combination. The process of the invention is preferably carried out continuously.

Before the work-up, the liquid product mixture is generally depressurized to a lower pressure level if the hydrogenation reaction has been carried out under superatmospheric pressure, for example to from 0.1 to 0.5 MPa absolute. Here, dissolved hydrogen-comprising gas is liberated and is either discharged or recirculated.

The liquid product mixture can subsequently be fractionated by known methods, preferably by distillation. In the case of industrial processes, the fractionation is preferably carried out continuously in a plurality of columns. One work-up can, for example, be as follows: the liquid hydrogenation output goes into a first column (a) in which all THF together with water, preferably only part of the water which corresponds to the THF/water azeotrope at the pressure set, and other low boilers are separated off from the higher-boiling components at pressure of from 0.05 to 0.3 MPa absolute. The THF-comprising stream is fractionated further in a second column (b), preferably at pressures above that in the first column, for example at from 0.15 to 1.5 MPa absolute. The low-boiling THF/water azeotrope obtained here can be recirculated entirely or only partly to the first column. Low boilers present, e.g. methanol, can be discharged in their entirety or in part at the top of the column. Since these low boilers, for example methanol, still comprise THF, this mixture can, depending on the amount, be fractionated further in one or more separate column(s). The high-boiling product from column (b), viz. THF, can be saleable as such or, if purities above 99.9% are wanted, be subjected to fine purification in a further column.

One variant of this work-up is, especially when methanol contents of >0.2% by weight are present in the hydrogenation output, to accumulate the methanol in the column combination (a)+(b) by recirculation of a THF/methanol-comprising low boiler stream from (b) to (a) until no more water goes over together with the methanol/THF-azeotrope from column (a) into the column (b). As a result, all the water is discharged together with excess methanol from the bottom of the column (a).

If products of value, e.g. 1,4-butanediol and/or gamma-butyrolactone, are still comprised, these can, as described below, either be isolated in pure form or be recirculated to the reaction after water and other undesirable components such as propanol and butanol have been separated off. A preferred variant is, if THF is the preferred product, to treat the high boiler stream from column (a), should it still comprise 1,4-butanediol, by means of an acidic catalyst in such a way that butanediol is cyclized to THF. This can be carried out directly using the high-boiling stream from column (a) or else after removal of components so that the butanediol is present in concentrated form. Catalysts for this cyclization can be homogeneously dissolved or be in heterogeneous form as suspension or as a fixed bed. The cyclization can be carried out in the gas or liquid phase over fixed-bed catalysts, otherwise in the liquid phase. The temperatures here are in the range from 80 to 300° C., preferably from 90 to 250° C. Possible catalysts are inorganic acids such as sulfuric acid, phosphoric acid or heteropolyacids such as tungstophosphoric acid, organic acids such as sulfonic acids, acidic ion-exchange resins and acidic, inorganic solids such as zeolites, metal oxides such as SiO₂ or Al₂O₃, metal mixed oxides such as montmorillonites.

The high-boiling product from column (a) can, if it still comprises BDO and/or GBL which are to be recovered, be fractionated further, in a third column (c). Here, water and alcohols such as methanol, propanol and butanol are separated off as low boilers at pressures in the range from 0.01 to 1 MPa absolute, while a mixture comprising GBL and BDO is obtained as high boiler. This is fractionated in a 4th column (d) in such a way that GBL is obtained as low boiler and BDO is obtained as high boiler. Both product streams can be pure enough for sale, but can be subject to fine purification in further columns in each case.

The quality of the products is guided by the usual market standards. Thus, the products satisfy the usual GC purities, color numbers and other indices.

The invention is illustrated by the following examples.

### EXAMPLEs

The analysis of the product was carried out by gas chromatography. The product compositions reported are all calculated on a water-free basis. The 1,4-butynediol used was prepared by reacting acetylene with formaldehyde over Cu/Bi catalysts by the method of Reppe. The technical-grade 1,4-butynediol used comprised about 50% by weight of butynediol, about 47% by weight of water, about 1% by weight of propynol and small amounts of further components such as cuprenes, Cu compounds, salts such as sodium formate. The pure 1,4-butynediol was in the form of a 40% strength aqueous solution. The conversion reported is of the 1,4-butynediol used. The selectivity figures take account of 1,4-butynediol still to be hydrogenated and intermediate. Here, 1,4-butenediol, 4-hydroxybutyraldehyde and acetals derived therefrom were taken into account as intermediates still to be hydrogenated or reacted to form THF, GBL or BDO.

### Examples 1 to 6

Examples 1-6 below were carried out using pure 1,4-butynediol as 40% strength by weight aqueous solution at atmospheric pressure. The butynediol solution was pumped continuously into an externally heated tube (diameter 2.7 cm). The tube was charged with glass rings (30 ml) in the upper region. This zone served as vaporization section in which 1,4-butynediol/water and hydrogen (300 liter/h) were heated and passed in gaseous form into a second zone in the reactor tube in which the catalyst was located (20 ml). After the reactor tube, the gaseous reactor output was cooled to about 20° C. and product which condensed out was collected. The offgas was passed through a cold trap at −78° C. and further product was condensed out in this way. The two condensates were combined for the purposes of analysis. The catalysts were activated in a stream of hydrogen before the reaction. The results are shown in table 1.

TABLE 1

| | Space velocity | | | | | | |
|---|---|---|---|---|---|---|---|
| | over the catalyst | | THF¹ | BDO² | GBL³ | BYD | |
| | (kg of butynediol/ | Temperature | (% by | (% by | (% by | conversion | Selectivity |
| Example/catalyst | liter of catalyst × h) | (° C.) | weight) | weight) | weight) | (%) | (%) |
| 1 | 0.5 | 225 | 47.3 | 16.3 | 1.6 | 99.8 | 65.5 |
| 5%Pd/C | | | | | | | |
| 2 | 0.5 | 220 | 0.5 | 48.3 | 4.1 | 98.1 | 77.6 |
| 0.6%Pd/SiO₂ | 1 | 220 | 0.3 | 38 | 6 | 99.9 | 79.9 |
| | 0.5 | 240 | 0.2 | 33.8 | 3 | 99.7 | 90.5 |
| 3 | 0.5 | 220 | 1.4 | 17.4 | 4 | 99.8 | 40.4 |
| 1%Rh/Al₂O₃ | 1 | 220 | 1.2 | 10.4 | 4 | 99.8 | 39.3 |
| | 0.5 | 240 | 0.6 | 4 | 1.9 | 89.3 | 62.6 |
| 4 | 0.5 | 220 | 16.5 | 1.4 | 12.1 | 96.6 | 35.2 |
| 5%Ni/TiO₂ | 1 | 220 | 2.1 | 6.6 | 1.6 | 86.1 | 62.5 |
| 5 | 0.5 | 220 | 2.6 | 0.2 | 33.6 | 89.3 | 44 |
| CuO/Al₂O₃ | 1 | 220 | 3.5 | 0.3 | 44.4 | 98.2 | 51.6 |
| 6 | 0.5 | 220 | 4.5 | 2.4 | 13.4 | 70.2 | 62.4 |
| 25%Cu/SiO₂ | 1 | 220 | 1.8 | 1.4 | 8.1 | 45.9 | 70.6 |

¹THF = tetrahydrofuran
²BDO = 1,4-butanediol
³GBL = gamma-butyrolactone
⁴BYD = 1,4-butynediol

### Examples 7 to 9

Examples 7 to 9 below were carried out using technical-grade 1,4-butynediol at atmospheric pressure. The 1,4-butynediol solution was pumped continuously into a thin film evaporator with about 99.5% by weight of the feed solution being fed in vapor form to the in each case about 50 ml of catalyst or mixture of two catalysts in a reactor tube having a diameter of 2.7 cm. Both reactor tube and thin film evaporator were operated together using recycle gas. The amount of fresh gas was 2.5 mol of hydrogen/mole of butynediol. After the reactor tube, the gaseous reactor output was cooled to about 20° C. and product which condensed out was collected. The offgas was passed through a cold trap at −78° C. and further product was condensed out in this way. The two condensates were combined for the purposes of analysis. The catalysts were activated in a stream of hydrogen before the reaction. The results are shown in table 2.

TABLE 2

| | Space velocity | | | | | | |
|---|---|---|---|---|---|---|---|
| | over the catalyst | | THF¹ | BDO² | GBL³ | BYD | |
| | (kg of butynediol/ | Temperature | (% by | (% by | (% by | conversion⁴ | Selectivity |
| Example/catalyst | liter of catalyst × h) | (° C.) | weight) | weight) | weight) | (%) | (%) |
| 7 | 0.5 | 180 | 10 | 67 | 5 | 100 | 89 |
| 5%Pd/C | | | | | | | |
| 8 | 0.3 | 180 | 10 | 51 | 4 | 100 | 91 |
| 25%Ni/10%Cu/ | | | | | | | |
| 2%MnOₓ/SiO₂ | | | | | | | |
| 9 | 0.7 | 220 | 5 | 26 | 50 | 99.9 | 95 |
| 51%Ni/17%Cu/ | 0.6 | 180 | 3 | 40 | 26 | 99.9 | 97 |
| 2%MnOₓ/ZrO₂ | 0.6 | 250 | 8 | 13 | 60 | 99.9 | 94 |

¹THF = tetrahydrofuran
²BDO = 1,4-butanediol
³GBL = gamma-butyrolactone
⁴BYD = 1,4-butynediol

### Examples 10 to 14

In these examples, aqueous pure or technical-grade butynediol (denoted by *) was vaporized in a stream of hydrogen under superatmospheric pressure in a vaporizer which comprised metal packing rings and was externally heated by means of oil (about 240° C.) and hydrogenated in a reactor tube filled with catalyst or catalyst mixture (100 ml unless indicated otherwise). The reactor tube was configured as a double-walled tube which was heated or cooled externally by means of oil. The reaction output was cooled and the product which condensed out was depressurized to atmospheric pressure through a valve, while the gas phase was recirculated via the vaporizer by means of a recycle gas blower. A small part of the gas was discharged as offgas. Hydrogen was introduced into the reaction in an amount corresponding to hydrogen consumed by reaction and offgas via a fresh gas supply. The reaction pressure was kept constant in this way. The experimental results are shown in table 2. The conversion of butynediol was quantitative in each case. To calculate the selectivity, products such as 1,4-butenediol and 4-hydroxybutanal and acetals thereof, gamma-butyrolactone (GBL) and butanediol (BDO) were included in the calculation as compounds still to be hydrogenated or reacted to form THF. The results are shown in table 3.

TABLE 3

| | Space velocity | | | | | | |
|---|---|---|---|---|---|---|---|
| | over the catalyst | | | THF¹ | BDO² | GBL³ | |
| | (kg ofbutynediol/ | Temperature | Pressure | (% by | (% by | (% by | Selectivity |
| Example/catalyst | liter of catalyst × h) | (° C.) | MPa | weight) | weight) | weight) | (%) |
| 10 | 0.2 | 220-230 | 0.5 | 75 | 15 | 1 | 93 |
| 0.5%Pd/C | | | | | | | |
| 11 | 0.2 | 220-230 | 0.9 | 79 | 11 | 1 | 92 |
| 0.5%Pd/ZrO₂ | | | | | | | |
| 12 | 0.3 | 225-240 | 0.9 | 82 | 10 | 0.2 | 94 |
| 0.5%Pd/C (20 ml) + | | | | | | | |
| 0.5%Pd/ZrO₂ (40 ml) | | | | | | | |
| 13* | 0.1 | 220-225 | 0.5 | 80 | 0 | 3 | 85 |
| 0.5%Pd/ZrO₂ (90 ml) + | | | | | | | |
| 20 ml Al2O3 | | | | | | | |
| 14 | 0.2 | 220-225 | 0.5 | 20 | 2 | 36 | 80 |
| 66%Co/20%Cu/ | | | | | | | |
| 7%MnOₓ/4%MoO_{y} | | | | | | | |

### Reworking of the Examples of DE-A 2029557

### Example 6 of DE-A 2029557

140 g of 1,4-butynediol in 360 g of water were hydrogenated in the liquid phase at 10.5 MPa and 275° C. over a catalyst comprising 5% of palladium on activated aluminum oxide as described in example 6 of DE-A 2029557. Only decomposition products and water could be found in the hydrogenation output. THF was not found.

### Examples 2, 3 and 4 of DE-A 2029557

Butynediol was hydrogenated as described in examples 2, 3 and 4 of DE-A 2029557. The hydrogenation output from the reworking of example 2 using Pd on activated aluminum oxide spheres as catalyst comprised <3% by weight of THF together with mainly n-butanol and water. In the case of Ni- and Ru-comprising catalysts as per examples 3 and 4, only water was found in the output. In the offgas, a little (<1%) of THF together with mainly hydrocarbons from methane to butane were detected.

### Comparative Example Based on DE-A 2029557

In a manner analogous to example 6 of DE-A 2029557, a 40% strength by weight aqueous butynediol solution was hydrogenated in the liquid phase over a catalyst comprising 5% of palladium on activated aluminum oxide (5% Pd/Al₂O₃) at 225° C. and 10.3 MPa. Apart from polymeric residues, many components were present in the liquid hydrogenation output. About 14% by weight of n-propanol, 10% by weight of n-butanol and 20% by weight of THF were found. The total proportion of organic constituents in the hydrogenation output corresponded to 10% of the amount of butynediol originally used. The selectivity to THF was only 2%.

### Example 6

A 40% strength aqueous butynediol solution was hydrogenated according to the invention in the gas phase over a catalyst comprising 5% of palladium on activated aluminum oxide (5% Pd/Al20₃) at an inlet temperature of 225° C. and 0.9 MPa. 75% by weight of THF, 20% by weight of n-butanol and less than 1% by weight each of gamma-butyrolactone, 1,4-butanediol and n-propanol were found in the hydrogenation output. The selectivity to THF was 75%, since the conversion of the butynediol was complete.

1. A process for preparing tetrahydrofuran, 1,4-butanediol and/or gamma-butyrolactone by hydrogenation of 1,4-butynediol, wherein 1,4-butynediol is vaporized in a hydrogen-comprising gas stream and hydrogenated in gaseous form over at least one catalyst which comprises at least one of the elements of groups 7 to 11 of the Periodic Table of the Elements.

2. The process according to claim 1, wherein the temperature at which 1,4-butynediol is vaporized and the hydrogenation temperature are from 160 to 300° C.

3. The process according to either claim 1 or 2, wherein the pressure at which 1,4-butynediol is vaporized corresponds to at least the pressure of the subsequent hydrogenation.

4. The process according to any of claims 1 to 3, wherein the hydrogenation is carried out at a pressure of from 0.5 to 10 MPa.

5. The process according to any of claims 1 to 4, wherein the temperature at which 1,4-butynediol is hydrogenated is from 160 to 300° C.

6. The process according to any of claims 1 to 5, wherein the catalyst comprises at least one element selected from among Mn, Re, Fe, Ru, Co, Rh, Ir, Ni, Pd, Pt, Cu and Au.

7. The process according to any of claims 1 to 6, wherein the catalyst comprises Pd, Ni and/or Cu as active metal of groups 7 to 11 of the Periodic Table of the Elements.

8. The process according to any of claims 1 to 7, wherein the catalyst(s) comprises a support selected from among the oxides of aluminum, titanium, zirconium oxide, silicon oxide, clay minerals, silicates, zeolites and activated carbon.

9. The process according to any of claims 1 to 8, wherein an acidic catalyst without hydrogenation properties is additionally used.

10. The process according to any of claims 1 to 9, wherein the molar ratio of hydrogen to 1,4-butynediol fed in is at least 2 during the hydrogenation.

11. The process according to any of claims 1 to 10, wherein the hydrogenation is carried out using the gas recycle mode.
